(19) United States
(12) Patent Application Publication
(10) Pub. No.: US 2011/0097793 A1
(43) Pub. Date: Apr. 28, 2011

(54) CELL SEPARATION DEVICE, CELL SEPARATION SYSTEM AND CELL SEPARATION METHOD

(75) Inventors: Yuji Suzuki, Chofu-shi (JP); Nobuhide Kasagi, Tokyo (JP); Takahiro Nishimura, Fukuoka-shi (JP); Junichi Miwa, Ryugasaki-shi (JP)

(73) Assignee: THE UNIVERSITY OF TOKYO, Tokyo (JP)

(21) Appl. No.: 12/994,802

(22) PCT Filed: May 27, 2009
(86) PCT No.: PCT/JP2009/002335

§ 371 Date: Dec. 30, 2010

(30) Foreign Application Priority Data
May 30, 2008 (JP) 2008-142742
Jan. 23, 2009 (JP) 2009-012777

(51) Int. Cl. C12N 5/02; C12M 1/00

(52) U.S. Cl. 435/325; 435/283.1

(57) ABSTRACT

A cell separation device which can perform a continuous processing without bonding fluorescent molecules or magnetic particles to the surface of the cell membrane, a cell separation system, and a cell separation method, wherein when a sample cell suspension containing the desired target cells is supplied continuously from a sample inlet and physiological saline is supplied continuously from a physiological saline inlet, the sample cell suspension flows together with the physiological saline in a liquid flow path and an adsorption force acts on the target cells due to affinity bonding from the adsorbing portions of adsorbing regions in the form of strips formed in a planar wall portion. Since the adsorbing regions in the form of strips are disposed in an asymmetric fashion to the flow path direction of the liquid flow path, the adsorption force acting on the target cells has a constituent perpendicular to the flow path direction. As a result, the target cells shown in FIG. 1 collect on one side of the planar wall portion after flowing for a prescribed distance in the liquid flow path and can be separated continuously from the non-target constituents.

### BACKGROUND

The present invention relates to a cell separation device, a cell separation system, and a cell separation method to separate desired cells such as stem cells from a sample cell suspension and recover them.

RELATED ART

Conventionally, various techniques for separating and recovering stem cells contained in a sample cell suspension have been proposed. Examples of the technique for separating stem cells include a fluorescence activated-cell separation (FACS) procedure, an immunomagnetic cell separation (IMCS) procedure, and an immunoadsorption procedure. An example of IMCS is disclosed in Japanese Patent Application Laid-Open (JP-A) No. 2006-6166. An example of the immunoadsorption procedure is disclosed in Non-patent document below.

[Non-patent document] ADHESION-BASED CELL SORTER WITH ANTIBODY-IMMOBILIZED FUNCTIONALIZED-PARYLENE SURFACE Proc. IEEE Int. Conf. MEMS 2007, Kobe (2007), pp. 27-30 Junichi Miwa, et al.

However, in the case of FACS or IMCS, since fluorescent molecules or magnetic particles are bonded to the surface of the cell membrane of cells after separation, the safety of returning cells to which the fluorescent molecules or the magnetic particles are bonded to the body is unknown when it is a given fact that separated cells like stem cells are returned to the body. In the case of the immunoadsorption procedure, it is necessary to perform batch processing, and thus it is not suitable for high-throughput processing.

### SUMMARY

The present invention has been achieved in view of the above technical problems. An objective of the present invention is to provide a cell separation device, a cell separation system, and a cell separation method which can perform a continuous processing without bonding fluorescent molecules or magnetic particles to the surface of the cell membrane.

According to a first aspect of the invention, there is provided a liquid flow path that passes a liquid containing predetermined cells, a planar wall portion that is formed on at least a part of the inner wall surface of the liquid flow path, and adsorbing regions in the form of strips in which adsorbing portions having adsorptive properties to the predetermined cells because of affinity bonding to the surface of the predetermined cells are formed in the form of strips in the planar wall portion and disposed in an asymmetric fashion to the flow path direction.

According to a second aspect of the invention, there is provided a cell separation device in which a plurality of the adsorbing regions in the form of strips are disposed on the planar wall portion in the cell separation device according to the first aspect.

According to a third aspect of the invention, there is provided a cell separation device in which the adsorbing portions are formed using antibodies specifically bonded to antigens present in the cell membrane surface of the predetermined cells in the cell separation device according to the first or second aspect.

According to a fourth aspect of the invention, there is provided a cell separation device in which the adsorbing regions in the form of strips are formed by forming concave portions and convex portions alternatively disposed in an asymmetric fashion to the flow path direction of the liquid flow path on the planar wall portion and forming the adsorbing portions on at least the convex portions in the cell separation device according to any one of the first to third aspects.

According to a fifth aspect of the invention, there is provided a cell separation device in which the form of the adsorbing regions in the form of strips is a linear form in the cell separation device according to any one of the first to fourth aspects.

According to a sixth aspect of the invention, there is provided a cell separation device in which the form of the adsorbing regions in the form of strips is a staircase pattern in the cell separation device according to any one of the first to fourth aspects.

According to a seventh aspect of the invention, there is provided a cell separation device in which the form of the adsorbing regions in the form of strips is a wavelike form in the cell separation device according to any one of the first to fourth aspects.

According to a eighth aspect of the invention, there is provided a cell separation device in which one or a plurality of adsorbing portions in a predetermined form which have outline portions disposed in an asymmetric fashion to the flow path direction are provided in place of the adsorbing regions in the form of strips in the cell separation device according to any one of the first to fourth aspects.

According to a ninth aspect of the invention, there is provided a cell separation system in which a plurality of the cell separation devices according to any one of the first to eighth aspects are arranged in series.

According to a tenth aspect of the invention, there is provided a cell separation method which includes the steps of forming a planar wall portion in a planar form in at least a part of the inner wall surface of the liquid flow path, forming adsorbing portions having adsorptive properties to the predetermined cells because of affinity bonding to the surface of the predetermined cells in the form of strips in the planar wall portion, disposing them in an asymmetric fashion to the flow path direction, and passing a liquid containing the predetermined cells to the liquid flow path.

### BRIEF DESCRIPTION OF THE DRAWINGS

Exemplary embodiments of the present invention will be described in detail based on the following figures, wherein:

FIG. 1 is a schematic diagram of a structural example of the cell separation device according to the embodiment;

FIG. 2 is an explanatory diagram of the method for immobilizing antibodies in the adsorbing regions in the form of strips;

FIG. 3 is a diagram showing examples of sectional shapes of the liquid flow paths;

FIG. 4 is a schematic diagram of another structural example of the cell separation device according to the embodiment;

FIG. 5 is a diagram showing examples of shapes of the adsorbing regions in the form of strips;

FIG. 6 is a diagram showing examples of the adsorbing portions which can be used instead of the adsorbing regions in the form of strips;

FIG. 7 is a schematic diagram of a structural example of the cell separation system according to the embodiment;

FIG. 8 is a process chart of a production method of the cell separation device according to the embodiment;

FIG. 9 is a diagram showing the measurement results of the position in the direction perpendicular to the flow path direction and number of each of the mimic particles at a distance of 20 mm passed through the planar wall portion of the liquid flow path;

FIG. 10 is a diagram showing the measurement results of the position in the direction perpendicular to the flow path direction and number of human umbilical vein endothelial cells at a distance of 20 mm passed through the planar wall portion of the liquid flow path.

### DETAILED DESCRIPTION OF EXEMPLARY EMBODIMENTS

An exemplary embodiments of the present invention (hereinafter referred to as an embodiment) will be described with reference to drawings.

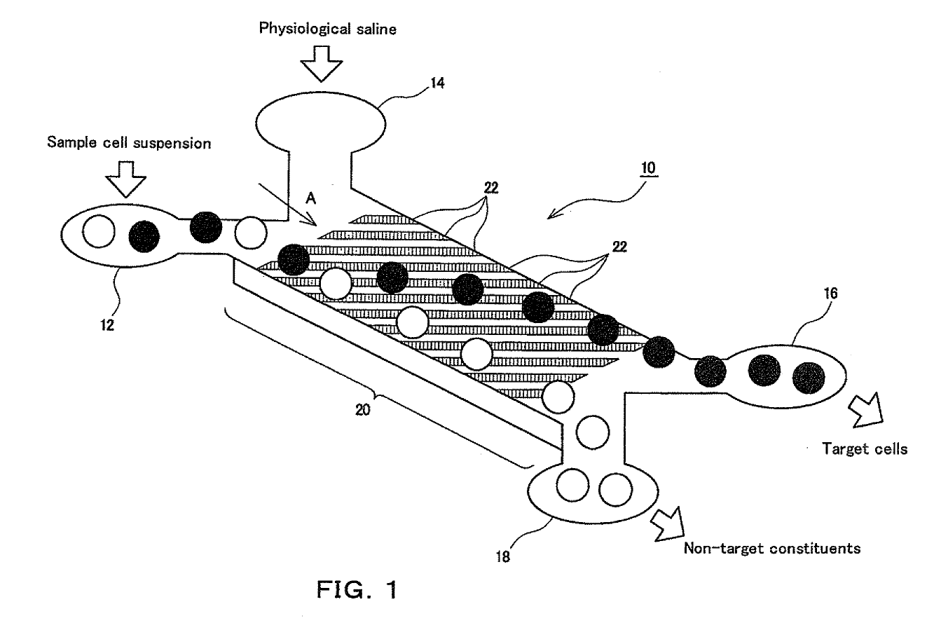

A schematic diagram of a structural example of the cell separation device according to the embodiment is shown in FIG. 1. In FIG. 1, a sample inlet 12 that supplies a sample cell suspension, a physiological saline inlet 14 that supplies physiological saline, a target cell discharging outlet 16 that collects target cells, and a non-target constituent outlet 18 that discharges constituents other than the target cells are provided in a liquid flow path 10. In the example of FIG. 1, target cells are indicated by black circles and non-target constituents (cells etc.) are indicated by white circles.

A planar wall portion 20 in a planar form is formed on at least a part of the inner wall surface of the liquid flow path 10. The planar wall portion 20 is not necessarily to have a perfect planar surface, it may be in a nearly planar form. Any material for constituting the planar wall portion 20 may be used, as long as it has a functional group for introducing adsorbing portions, such as antibodies, into the surface. For example, a material prepared by uniformly vapor-depositing a polyparaxylylene resin (diX-AM, manufactured by KISCO, Inc.) having an aminomethyl group (—CH₂—NH₂) on the surface of a glass substrate can be used. The adsorbing portions have adsorptive properties to the predetermined cells because of affinity bonding to the surface of the predetermined target cells. This can be formed using, for example, antibodies specifically bonded to antigens present in the cell membrane surface of the predetermined target cells. As for the liquid flow path 10 shown in FIG. 1, it is in a hollow form in which the cross section including the planar wall portion 20 is closed, while only the planar wall portion 20 is described because of the convenience of explanation. Further, a part of the cross section may have an opening. The sectional shape of the liquid flow path 10 will be described below.

In the embodiment, adsorbing regions 22 in the form of strips in which the adsorbing portions in the form of strips are fixed to the planar wall portion 20 are formed. A plurality of the adsorbing regions 22 in the form of strips are disposed in an asymmetric fashion to the flow path direction (flow direction) of the liquid flow path 10 at predetermined intervals. Here, the flow direction is a direction indicated by an arrow A in FIG. 1 as well as a longitudinal direction of the liquid flow path 10. Further, the term “asymmetric fashion” means an inclination at a predetermined angle to the flow path direction as shown in FIG. 1. The angle of inclination in this case affects the displacement of the target cells to a direction perpendicular to the flow path direction while flowing through the liquid flow path 10. Further, the width of the planar wall portion 20, the width of the adsorbing regions 22 in the form of strips and mutual intervals between the adsorbing regions 22 in the form of strips are appropriately determined based on the size of the target cells.

The adsorbing regions 22 in the form of strips constituted by the adsorbing portions are formed by fixing, for example, predetermined antibodies to functional groups present on the surface of the planar wall portion 20. In this case, the functional groups of the surface of the planar wall portion 20 are patterned so that antibodies are fixed to the planar wall portion 20 in the form of strips at predetermined intervals. Then, the adsorbing regions 22 in the form of strips are formed by a method for immobilizing the antibodies to the functional groups. In this regard, a method of removing an unnecessary portion of the functional groups by oxygen plasma after masking with photoresist can be used in patterning of the functional groups.

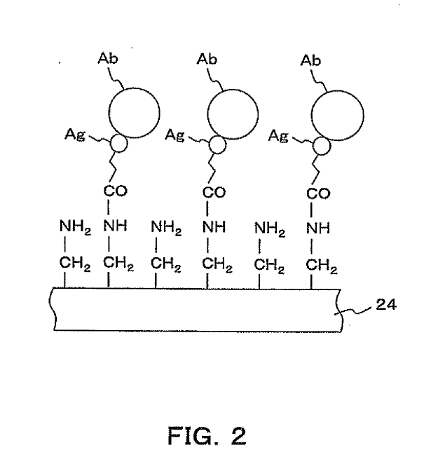

An explanatory diagram of the method for immobilizing antibodies in the adsorbing regions 22 in the form of strips is shown in FIG. 2. In FIG. 2, an aminomethyl group (—CH₂—NH₂) of the polyparaxylylene resin (parylene) is present on the surface of a substrate 24 such as glass and silicon. An antigen (Ag) to which a carboxy group is bonded is immobilized to the amino group by an acid-amide bond and an antibody Ab is bonded to the antigen Ag for immobilization.

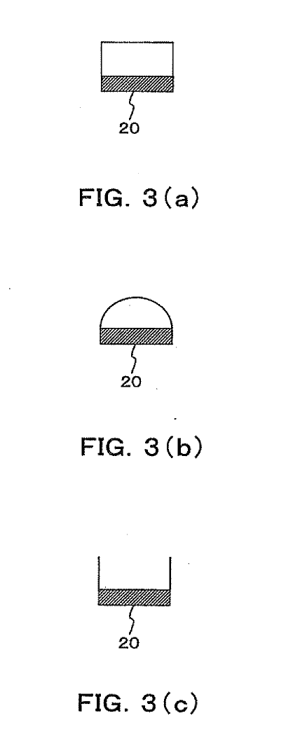

Examples of sectional shapes of the liquid flow paths 10 are shown in FIG. 3(a), (b), and (c). In the example of FIG. 3(a), the cross section except the planar wall portion 20 is rectangular. In the example of FIG. 3(b), the cross section except the planar wall portion 20 has an arch-form. In the example of FIG. 3(c), a part of the cross section except the planar wall portion 20 is opened. However, the sectional shape of the liquid flow path 10 is not limited thereto and it can be appropriately determined according to the target cells and the amount of throughput.

Returning to FIG. 1, a sample cell suspension containing the desired target cells is supplied continuously from the sample inlet 12 and physiological saline is continuously supplied from the physiological saline inlet 14. The sample cell suspension flows together with the physiological saline in the liquid flow path 10 and an adsorption force acts on the target cells (black circles) due to affinity bonding from the adsorbing portions of the adsorbing regions 22 in the form of strips formed in the planar wall portion 20. The affinity bonding is based on, for example, an antigen antibody interaction, an interaction between ligands and receptors, an interaction of biotin and avidin, an interaction due to charges (positive charge and negative charge), and an interaction between sugar chain and lectin. As described above, since the adsorbing regions 22 in the form of strips are disposed in an asymmetric fashion to the flow path direction of the liquid flow path 10 and have an inclination at a predetermined angle, the adsorption force acting on the target cells has a constituent perpendicular to the flow path direction. As a result, the target cells shown in FIG. 1 collect on one side of the planar wall portion 20 after flowing for a prescribed distance in the liquid flow path 10. In order to separate the target cells, it is preferable to adjust the inclination angle of the adsorbing regions 22 in the form of strips so that the side where the target cells collect is the side opposite to the sample inlet 12 in the planar wall portion 20. Thus, the target cells collected at one side of the planar wall portion 20 are taken out continuously from the target cell discharging outlet 16 provided at the same side.

On the other hand, an adsorption force from the adsorbing portions of the adsorbing regions 22 in the form of strips does not act on the non-target constituents (white circles). Then, the non-target constituents flow through the liquid flow path 10 at the position flowed from the sample inlet 12, namely, they does not move to the direction perpendicular to the flow path direction. As a result, they are discharged continuously from the non-target constituent outlet 18 provided at the same side of the sample inlet 12 of the planar wall portion 20.

As the results above, the target cells contained in the sample cell suspension that is supplied from the sample inlet 12 can be separated continuously from non-target constituents. In the process, it is not necessary to bind fluorescent molecules or magnetic particles to the surface of the cell membrane.

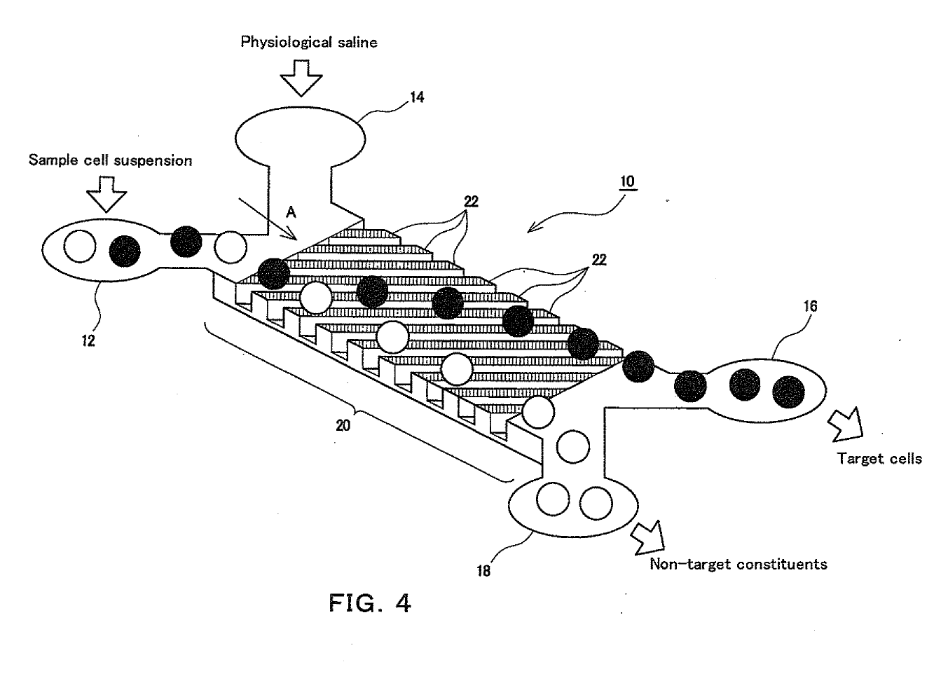

A schematic diagram of another structural example of the cell separation device according to the embodiment is shown in FIG. 4 and the same symbols are given to the same elements as FIG. 1. In FIG. 4, a characteristic point is that concave portions and convex portions alternatively disposed in an asymmetric fashion to the flow path direction of the liquid flow path 10 on the planar wall portion 20 are formed. In this regard, the planar wall portion 20 in the embodiment is in the planar form in the sense that the concave portions and the convex portions are formed on a smooth planar surface. Further, adsorbing portions in which predetermined antibodies are fixed are provided on at least the top planar surfaces of convex portions to form the adsorbing regions 22 in the form of strips. In this regard, the adsorbing portions may be provided on the bottom surfaces of the concave portions and the side wall that connects the top planar surface of the convex portions to the bottom surface of the concave portions.

In the liquid flow path 10 shown in FIG. 4, the inclination angle of the adsorbing regions 22 in the form of strips affects the displacement of the target cells to a direction perpendicular to the flow path direction while flowing through the liquid flow path 10. Further, the width of the convex portions of the adsorbing regions 22 in the form of strips and mutual intervals between the convex portions are appropriately determined based on the size of the target cells. In this regard, the width of the convex portions of the adsorbing regions 22 in the form of strips and the mutual intervals of the convex portions are set so that the passing target cells may not contact the bottom of the concave portions.

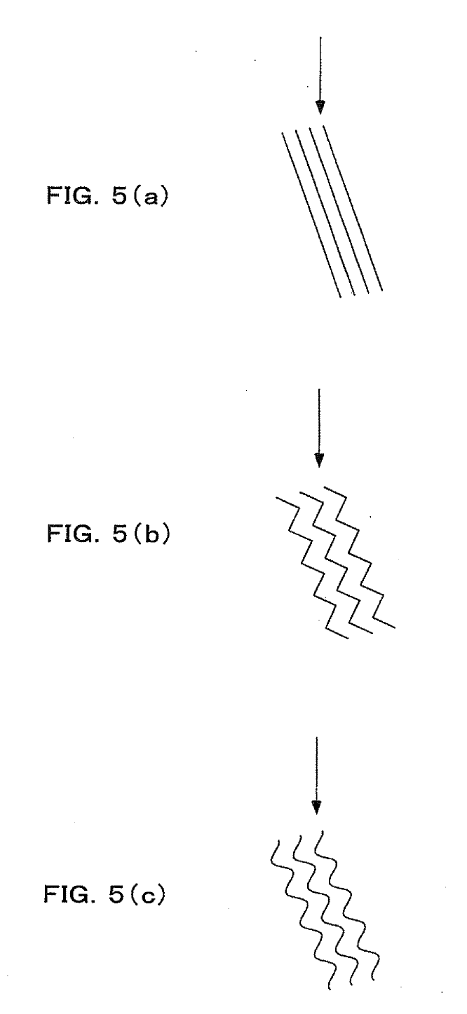

Examples of shapes of the adsorbing regions 22 in the form of strips described above are shown in FIGS. 5(a), (b), and (c). In the example of FIG. 5(a), a plurality of adsorbing regions 22 in the form of strips in an asymmetric fashion to the flow path direction indicated by an arrow, namely, with an inclination at a predetermined angle are formed in a linear form. Their forms are the same as those of FIGS. 1 and 4. In the example, the forms of the adsorbing regions 22 in the form of strips are simply illustrated only by a straight line. Adsorbing portions are alternately provided in the regions divided by each straight line. The same holds true for FIGS. 5(b) and (c) described below. In the example of FIG. 5(b), a plurality of the adsorbing regions 22 in the form of strips are formed in a staircase pattern. In the example of FIG. 5(c), a plurality of the adsorbing regions 22 in the form of strips are formed in a wavelike form. However, the shape of the adsorbing regions 22 in the form of strips is not limited thereto and it can be appropriately determined according to the target cells and the amount of throughput.

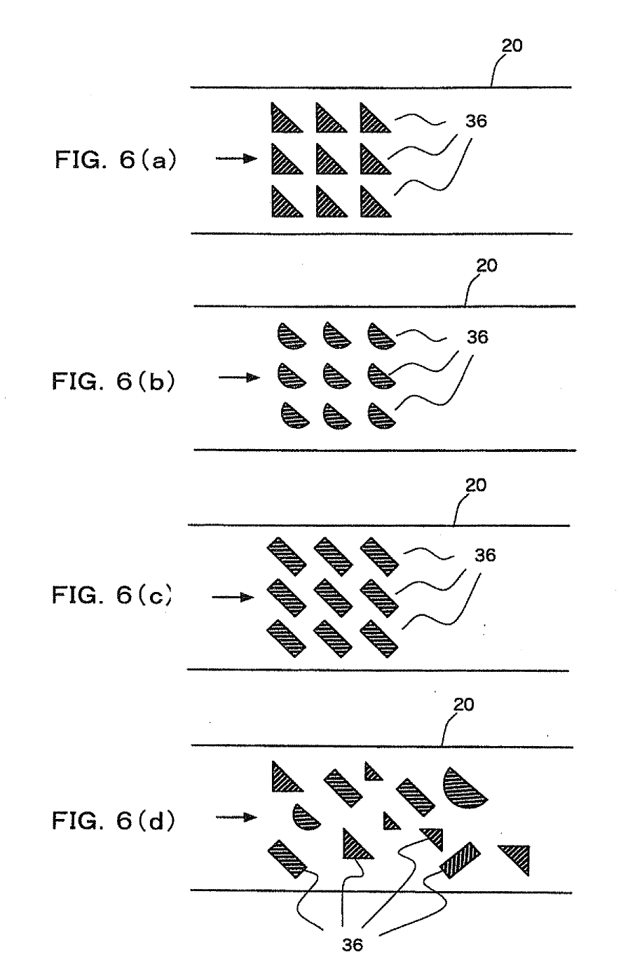

Examples of the adsorbing portions which can be used instead of the adsorbing regions 22 in the form of strips are shown in FIGS. 6(a), (b), (c), and (d). The flow path directions indicated by arrows are horizontal in the drawing.

In FIG. 6(a), a plurality of the adsorbing portions 36 in the form of a right triangle are disposed. Further, they are disposed with an inclination of a hypotenuse which is part of the outline portions of the right triangle, so as to be asymmetrical to the flow path direction. In the example, they are disposed so that the hypotenuse of the right triangle is located at the downstream in the flow path direction. However, they may be disposed so that the hypotenuse is located at the upstream side. Further, the adsorbing portions 36 are disposed in a line. However, they may be randomly disposed as long as the part of the outline portions is asymmetrical to the flow path direction. Further, they are disposed so that the outline portion (e.g. hypotenuse) asymmetrical to the flow path direction faces the side where the target cells are separated at the downstream side in the flow path direction, for example, the side where the target cell discharging outlet 16 of FIG. 1 is disposed.

In FIG. 6(b), a plurality of the adsorbing portions 36 in the form of an arch are disposed. Further, they are disposed with an inclination of a string which is part of the outline portions of the arch form, so as to be asymmetrical to the flow path direction. In the example, they are disposed so that the string is located at the downstream in the flow path direction. However, they may be disposed so that the string is located at the upstream side. Further, the adsorbing portions 36 are disposed in a line. However, they may be randomly disposed as long as the part of the outline portions is asymmetrical to the flow path direction. Further, they are disposed so that the outline portion (e.g. string) asymmetrical to the flow path direction faces the side where the target cells are separated at the downstream side in the flow path direction, for example, the side where the target cell discharging outlet 16 of FIG. 1 is disposed.

In FIG. 6(c), a plurality of the adsorbing portions 36 in the form of a rectangle are disposed. Further, they are disposed with an inclination of a side which is the outline portions of the rectangle, so as to be asymmetrical to the flow path direction. In the Example, the adsorbing portions 36 are disposed in a line. However, they may be randomly disposed as long as the part of the outline portions is asymmetrical to the flow path direction. Further, they are disposed so that the outline portion (e.g. long side) asymmetrical to the flow path direction faces the side where the target cells are separated at the downstream side in the flow path direction, for example, the side where the target cell discharging outlet 16 of FIG. 1 is disposed. In this case, a short side faces the side different from the long side at the downstream side in the flow path direction. The long side of the rectangle has a longer distance to transfer the target cells when compared with the short side. This allows the target cells to be transferred to a direction that separates the target cells from non-target constituents.

In FIG. 6(d), plural types of the adsorbing portions 36 are mixed and randomly disposed. In this case, the adsorbing portions 36 are disposed so that the total of the length of the outline portions asymmetrical to the flow path direction facing the side that the target cells are separated at the downstream side in the flow path direction is longer than the total of the length of the outline portions asymmetrical to the flow path direction facing the side different therefrom.

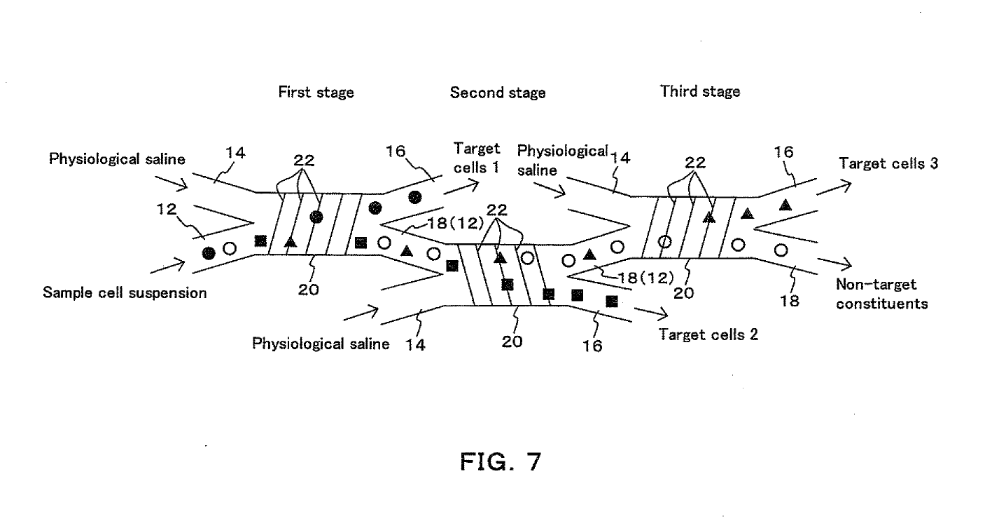

A schematic diagram of a structural example of the cell separation system according to the embodiment is shown FIG. 7. In FIG. 7, the cell separation device is connected in three-stage series. Here, the term “in series” means a process of connecting the non-target constituent outlet 18 of the cell separation device in a certain stage to the sample inlet 12 of the cell separation device in the next stage. According to such a structure, three types of target cells can be separated. Further, the adsorbing regions 22 in the form of strips formed on the planar wall portion 20 of each of the cell separation devices to be used herein have adsorptive properties to each of the target cells. In this case, it is preferable to select the adsorbing regions 22 in the form of strips of a certain cell separation device which have adsorptive properties to the target cells of the cell separation device, and has weak adsorptive properties or does not have adsorptive properties to the target cells separated by other cell separation devices. However, the regions having adsorptive properties to the target cells separated by other cell separation devices can be used depending on the type of target cells and convenience of the separation process.

In FIG. 7, when a sample cell suspension containing plural types of the target cells is supplied from the sample inlet 12 at the first stage of the cell separation device and physiological saline is supplied from the physiological saline inlet 14, target cells 1 are taken out from the target cell discharging outlet 16 of the cell separation device at the first stage by the operation of the adsorbing regions 22 in the form of strips formed in the planar wall portion 20. Further, constituents other than the target cells 1 as non-target constituents are supplied from the non-target constituent outlet 18 at the first stage of the cell separation device to the sample inlet 12 of the cell separation device at the second stage. In the second stage of the cell separation device, the target cells 2 are taken out from the target cell discharging outlet 16 of the cell separation device at the second stage in the same manner as described above by the operation of the adsorbing regions 22 in the form of strips formed in the planar wall portion 20. Further, constituents other than the target cells 2 as non-target constituents are supplied from the non-target constituent outlet 18 of the cell separation device at the second stage to the sample inlet 12 of the cell separation device at the third stage. In the cell separation device at the third stage, separation is performed in the same manner as described above. Target cells 3 are taken out from the target cell discharging outlet 16 of the cell separation device at the third stage. Constituents other than this, as non-target constituents, are discharged from the non-target constituent outlet 18 of the cell separation device at the third stage.

As described above, the cell separation system according to the embodiment shown in FIG. 7 can separate three types of target cells. In this regard, the number of stages in the cell separation device in the cell separation system is not limited to three stages and the number of stages can be determined according to the number of types of the target cells. Although the adsorbing regions 22 in the form of strips are used in the example shown in FIG. 7, the adsorbing portions 36 shown in FIG. 6(a), (b), (c), and (d) may be used.

According to each embodiment described above, the target cells can be separated even if markers, such as fluorescence particles (molecules) and magnetic particles are not used. Since the target cell can be separated only by pouring the sample cell suspension into the cell separation device, the external operation for separation and peripheral devices are completely unnecessary. Thus, the minimization is quite easy as compared with devices such as large FAGS and IMCS. Further, a pump for supplying the sample cell suspension or physiological saline and another power are unnecessary.

### EXAMPLES

Examples of the embodiments described above will be described hereinafter.

### Example 1

### Production of the Cell Separation Device

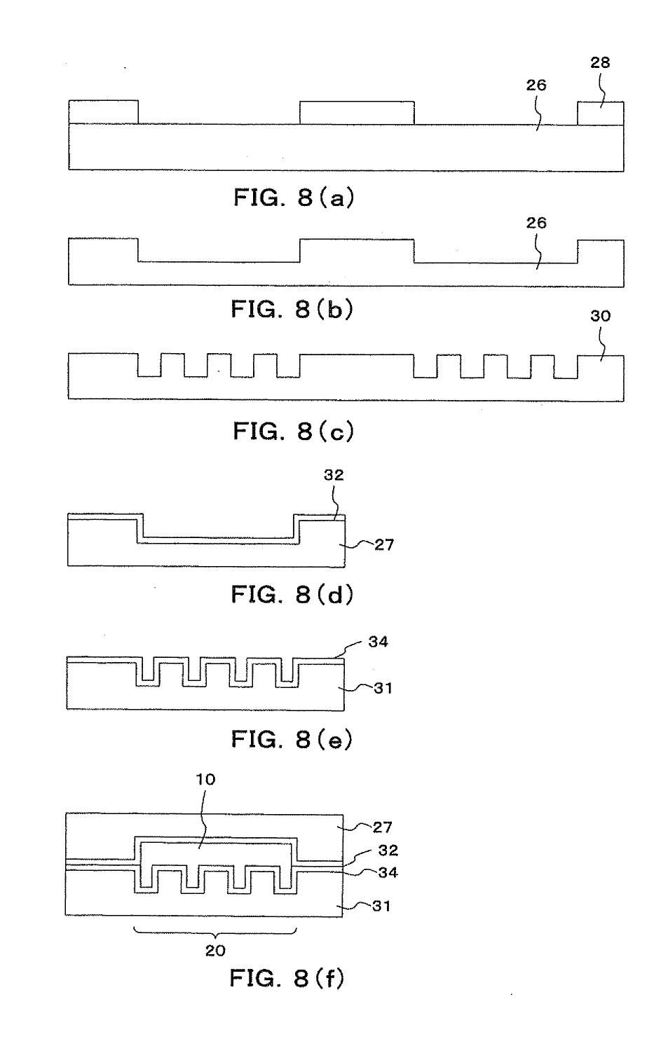

A process chart of a production method of the cell separation device according to the embodiment is shown in FIGS. 8(a) to (e). In FIG. 8(a), both sides of a silicon wafer 26 are thermally oxidized to form a silicon oxide film with a thickness of 200 nm. A positive type photoresist (AZP4400, manufactured by AZ Electronic Materials (Japan)) 28 is stacked on the silicon oxide film and the liquid flow path 10 is patterned by photolithography using a photomask having a flow path structure.

Subsequently, the silicon oxide film is patterned by dry etching using a photoresist as a mask with a high-density plasma etching device (CE-300I, manufactured by ULVAC, Inc.). At this time, CHF₃ is used as a process gas. Then, a passage groove being used as the liquid flow path 10, as shown in FIG. 8(b), is formed using the silicon oxide film as a mask by the reactive ion etching (Deep-RIE) according to the Bosch process which is an anisotropic dry etching process for silicon. The inductive coupling plasma reactive ion etching (ICP-RIE) device (AMS-100, manufactured by Alcatel) was used in the plasma etching. As for the process gas, SF₆ is used for etching, C₄F₈ is used to protect the side wall, and O₂ is used as an auxiliary gas for etching.

Further, as shown in FIG. 8(c), fine concavo-convex patterns (about 1 μm in wide) which are the adsorbing regions 22 in the form of strips are formed on the glass wafer 30 made of Pyrex (registered trademark) by electron-beam lithography. The fine concavo-convex patterns are formed in the form of strips and a plurality of the fine concavo-convex patterns are disposed with an inclination at a predetermined angle in the flow path direction of the liquid flow path 10 at predetermined intervals. Further, a chemical amplification type positive resist (FEP-171, manufactured by Fuji Photo Film Co., Ltd.) is used as an electron beam resist and the resist is spin-coated, followed by spin-coating of a conductive polymer (Espacer 300AX, manufactured by Showa Denko K.K.). After patterning the electron beam resist, dry etching (a depth of 250 nm) of Pyrex glass is performed.

The silicon wafer 26 and the glass wafer 30 made of Pyrex explained above after protecting their surfaces by photoresist are cut into 2×2 cm² pieces using a dicing saw (DAD340, manufactured by DISCO Corporation) to form a silicon substrate 27 and a glass substrate 31, respectively shown in FIG. 8(d). Thereafter, the sample inlet 12, the physiological saline inlet 14, the target cell discharging outlet 16, and the non-target constituent outlet 18 are provided on the silicon substrate 27 using an ultrasonic-working machine (manufactured by Ultrasonic Engineering Co., Ltd).

In FIG. 8(d), the silicon substrate 27 and the glass substrate 31 are washed carefully, followed by uniform vapor deposition of the parylenes (polyparaxylylene resin) 32 and 34 on the surfaces of the substrates using a parylene vapor deposition apparatus (PDS-2010, manufactured by Parylene Japan, LLC). For the parylene 32 of the silicon substrate 27, diX-C (manufactured by KISCO LTD.) is vapor-deposited at a thickness of 2 μm. For the parylene 34 of the glass substrate 31, diXAM (manufactured by KISCO LTD.) having an aminomethyl group (—CH₂—NH₂) is vapor-deposited at a thickness of 0.1 μm. Both of the substrates 27 and 31 in which the parylenes 32 and 34 are vapor-deposited are aligned with a microscope, and then they are bonded by thermo-compression using a nanoimprint apparatus (NI-273, manufactured by Nano Craft Technologies Co.). The thermocompression-bonding conditions at this time include a pressure of 10 Pa or less, a temperature of 150° C., a load of 2000 N, and a thermocompression-bonding time of 30 minutes. As a result, the liquid flow path 10 having the planar wall portion 20 is formed as shown in FIG. 8(e).

Then, streptoavidin (antibody mimic substance) is fixed to the planar wall portion 20 by the following procedures.

First, 1 mg/mL of a dimethylsulfoxide solution of NHS-LC-LC-biotin is dissolved in 1 mL of bicin buffer solution whose pH is adjusted to 8.5. The resulting solution is introduced into the liquid flow path 10 using a syringe and allowed to stand for 1 hour. Next, the inside of the liquid flow path 10 is washed with ultrapure water and a solution prepared by dissolving 1 mg of streptoavidin in 0.5 mL of phosphate buffer solution (PBS) with a pH of 7.4 is introduced thereto, which is allowed to stand for 30 minutes. Finally, the resulting solution is washed with PBS. As a result, streptoavidin is fixed to the concave-convex portion of the planar wall portion 20 via biotin and the adsorbing regions 22 in the form of strips are formed.

The cell separation device shown in FIG. 1 is completed by the process.

### Example 2

### Cell Separation Test

Fluorescent particles with biotin (excitation: 475 nm) were used as mimic particles of the target cells and fluorescent particles with streptoavidin (excitation: 520 nm) were used as mimic particles of non-target constituents. The sample cell suspension mixed with these mimic particles was supplied from the sample inlet 12 and physiological saline was supplied from the physiological saline inlet 14. When supplying these materials, a syringe pump (CMA400, manufactured by Microdialysis) was used. Each mimic particle (100 particles) passed near the target cell discharging outlet 16 and the non-target constituent outlet 18 was photographed using a monochrome cooled CCD camera (Rolera XR, manufactured by QImaging). The position in the direction perpendicular to the flow path direction on the planar wall portion 20 and the number of each mimic particle were measured.

In the example, the inclination angle of the adsorbing regions 22 in the form of strips used was 45 degrees. Further, photographs at a distance of 20 mm passed through the planar wall portion 20 of the liquid flow path 10 were taken with a monochrome cooled CCD camera. The width of the planar wall portion 20 was set to 200 μm.

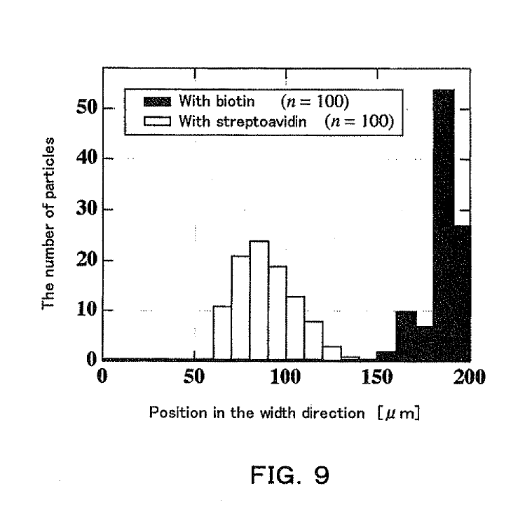

The measurement results are shown in FIG. 9. In FIG. 9, a horizontal axis is a position in the width direction of the planar wall portion 20. The term “position in the width direction” means a distance from the side of the sample inlet 12 in the width direction (direction perpendicular to the flow path direction) of the planar wall portion 20. A vertical axis is the number of particles.

As shown in FIG. 9, after passing a distance of 20 mm, the mimic particles (with biotin) of the target cells move 150 μm or more in the direction perpendicular to the flow path direction. On the other hand, the moving distance of the mimic particles of the non-target constituents (with streptoavidin) remains at 140 μm or less. Therefore, cells can be continuously separated by the cell separation device according to the example.

In Example 1 and Example 2 described above, the production of the cell separation device and the effect test were performed using the mimic particles in place of the real cells and the antibody mimic substance in place of antibodies. However, real cells and antibodies can also be used as shown in Example 3 below.

### Example 3

### Cell Separation Test Using Real Cells

The separation test was performed using human umbilical vein endothelial cells which are real cells as the target cells. The method for producing the cell separation device used in the process is described below.

First, the liquid flow path 10 having the planar wall portion 20 is formed in the same manner as described in FIG. 8(a) to (e) of Example 1. Next, streptoavidin is fixed to the concavo-convex portion of the planar wall portion 20 via biotin by the same process as Example 1.

Then, 1 μg/ml of human CD31 antibodies after biotin labeling is introduced into the liquid flow path 10, which is allowed to stand for 30 minutes, followed by washing with PBS. Thus, the CD31 antibodies can be fixed to streptoavidin fixed to the concavo-convex portion of the planar wall portion 20. As the result, the adsorbing regions 22 in the form of strips in which the adsorbing portions are comprised of the CD31 antibodies are formed.

Subsequently, human umbilical vein endothelial cells (the target cells) are stained with SYTO24 fluorescent dye (excitation: 490 nm). This staining process is performed for making confirmation of the separation of the target cells easy. Thus, the process is unnecessary when separating the target cells being used in the body by the cell separation device of the example.

The human umbilical vein endothelial cells after staining were dispersed in a phosphate buffer solution (PBS) with a pH of 7.4 so that the cell density was 2×10⁶ cells/ml to form a cell suspension. Then, the cell suspension was continuously introduced to the cell separation device in which the adsorbing regions 22 in the form of strips were formed with the CD31 antibodies from the sample inlet 12. PBS was continuously introduced from the physiological saline inlet 14. The flow ratio of the cell suspension to PBS in this case was 1 (cell suspension): 2.5 (PBS). The mean velocity of the bulk in the liquid flow path 10 was 1 mm/s. In this regard, the syringe pump (CMA400, manufactured by Microdialysis) was used in supplying from the sample inlet 12 and the physiological saline inlet 14. The target cells passed near the target cell discharging outlet 16 and the non-target constituent outlet 18 was photographed using a monochrome cooled CCD camera (Rolera XR, manufactured by QImaging). The position in the direction perpendicular to the flow path direction on the planar wall portion 20 and the number of the human umbilical vein endothelial cells (the target cells) after staining were measured.

In the example, the inclination angle of the adsorbing regions 22 in the form of strips used was 45 degrees. Further, photographs at a distance of 20 mm passed through the planar wall portion 20 of the liquid flow path 10 were taken with a monochrome cooled CCD camera. Further, the width of the planar wall portion 20 was 200 μm.

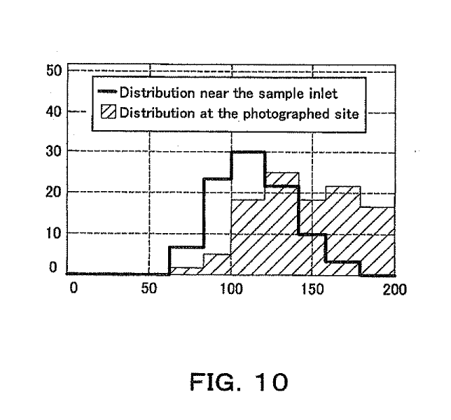

The measurement results are shown in FIG. 10. In FIG. 10, a horizontal axis is a position in the width direction of the planar wall portion 20. The term “position in the width direction” means a distance from the side of the sample inlet 12 in the width direction (direction perpendicular to the flow path direction) of the planar wall portion 20. A vertical axis is the number of cells. In FIG. 10, the distribution of the number of cells when the cell suspension is introduced from the sample inlet 12 is indicated by a solid line. The distribution of the number of cells which is analyzed from the image photographed with the monochrome cooled CCD camera is indicated by a shaded area. As shown in FIG. 10, when the cell suspension flows on the planar wall portion 20 in which the adsorbing regions 22 in the form of strips are formed, human umbilical vein endothelial cells (the target cells) move to the direction perpendicular to the flow path direction. Therefore, it is found that the target cells can be continuously separated by the cell separation device according to the example.

Although the exemplary embodiments of the invention have been described above, many changes and modifications will become apparent to those skilled in the art in view of the foregoing description which is intended to be illustrative and not limiting of the invention defined in the appended claims.

1. A cell separation device comprising:
a liquid flow path that passes a liquid containing predetermined cells;
a planar wall portion that is formed on at least a part of the inner wall surface of the liquid flow path; and
adsorbing regions in the form of strips in which adsorbing portions having adsorptive properties to the predetermined cells because of affinity bonding to the surface of the predetermined cells are formed in the form of strips in the planar wall portion and disposed in an asymmetric fashion to the flow path direction.

2. The cell separation device according to claim 1, wherein a plurality of the adsorbing regions in the form of strips are disposed on the planar wall portion.

3. The cell separation device according to claim 1, claim 1, wherein the adsorbing portions are formed using antibodies specifically bonded to antigens present in the cell membrane surface of the predetermined cells.

4. The cell separation device according to claim 1, wherein the adsorbing regions in the form of strips are formed by forming concave portions and convex portions alternatively disposed in an asymmetric fashion to the flow path direction of the liquid flow path on the planar wall portion and forming the adsorbing portions on at least the convex portions.

5. The cell separation device according to claim 1, wherein the form of the adsorbing regions in the form of strips is a linear form.

6. The cell separation device according to claim 1, wherein the form of the adsorbing regions in the form of strips is a staircase pattern.

7. The cell separation device according to claim 1, wherein the form of the adsorbing regions in the form of strips is a wavelike form.

8. The cell separation device according to claim 1, wherein one or a plurality of adsorbing portions in a predetermined form which have outline portions disposed in an asymmetric fashion to the flow path direction are provided in place of the adsorbing regions in the form of strips.

9. A cell separation system, wherein a plurality of the cell separation devices according to claim 1 are arranged in series.

10. A cell separation method comprising the steps of:
forming a planar wall portion in a planar form on at least a part of the inner wall surface of the liquid flow path;
forming adsorbing portions having adsorptive properties to the predetermined cells because of affinity bonding to the surface of the predetermined cells in the form of strips in the planar wall portion;
disposing them in an asymmetric fashion to the flow path direction; and
passing a liquid containing the predetermined cells to the liquid flow path.
